(19) United States
(12) Patent Application Publication
(10) Pub. No.: US 2023/0129721 A1
(43) Pub. Date: Apr. 27, 2023

(54) STABILIZER FOR RADIOPHARMACEUTICALS AND RADIOPHARMACEUTICAL COMPOSITION COMPRISING THE SAME

(71) Applicants: THE ASAN FOUNDATION, Seoul (KR); UNIVERSITY OF ULSAN FOUNDATION FOR INDUSTRY COOPERATION, Ulsan (KR)

(72) Inventors: Sang Ju LEE, Seoul (KR); Seung Jun OH, Seoul (KR); Dae Hyuk MOON, Seoul (KR); Jin-Sook RYU, Seoul (KR); Jae-Seung KIM, Seoul (KR); Minyung OH, Seoul (KR); Yong-il KIM, Seoul (KR); Jong Jin LEE, Seoul (KR); Sun Young CHAE, Seoul (KR); Dong Yun LEE, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 17/995,024

(22) PCT Filed: Jan. 19, 2021
(86) PCT No.: PCT/KR2021/000703

§ 371 Date: Sep. 29, 2022

(30) Foreign Application Priority Data
Mar. 31, 2020 (KR) 10-2020-0039472

(51) Int. Cl. A61K 51/04; A61K 47/22; A61K 47/24; A61K 47/20

(52) U.S. Cl. CPC A61K 51/048; A61K 47/22; A61K 47/24; A61K 47/20; A61K 51/0455

(57) ABSTRACT

The present invention relates to: a composition for stabilizing a radiopharmaceutical, the composition comprising a vitamin B compound as an active component; and a radiopharmaceutical composition comprising the same. Due to the inclusion of the vitamin B compound, it is possible to stabilize the radiochemical purity of the radioactive compound by inhibiting the radioactive degradation thereof at room temperature as well as at high temperature.

### TECHNICAL FIELD

The present invention is a composition for stabilizing a radiopharmaceutical, which includes a vitamin B compound as an active component, and a radiopharmaceutical composition including the same.

This application claims priority to and the benefit of Korean Patent Application No. 10-2020-0039472 filed in the Korean Intellectual Property Office (KIPO) on Mar. 31, 2020, and all the contents disclosed in the specification and drawings of the application are incorporated in this application.

### BACKGROUND ART

Radiopharmaceuticals are drugs that are used to diagnose or treat diseases when the radiopharmaceuticals are labeled with a radioactive isotope and administered into the human body. A radioactive isotope used in the radiopharmaceuticals is unstable and thus converted into a stable isotope while releasing radiation. In this case, the released radiation may be used to diagnose and treat diseases. Radiation includes α rays, β rays, γ rays, positrons (β+rays), and the like. Radioactive isotopes used in positron emission tomography include fluoride ([¹⁸F]), carbon ([¹C]), nitrogen ([¹³N]), oxygen ([¹⁵O]), gallium ([⁶⁸Ga]), and the like. Among them, when carbon ([¹¹C]) is used, it is the most ideal radiopharmaceutical having the same structure as over-the-counter medicines, but has a drawback in that the time taken to manufacture and distribute radiopharmaceuticals should be short due to its very short half-life of 20 minutes. On the contrary, it has been reported that fluoride ([¹⁸F]) is very suitable for performing positron emission tomography because it has a size similar to that of hydrogen, forms a stable bond with a carbon atom of an organic compound, is easily produced, and has an appropriate half-life (110 minutes) (Lasne, M. C.; Perrio, C.; Rouden, J.; Barre, L.; Roeda, D.; Dolle, F.; Crouzel, C. Contrast Agents II, Topics in Current Chemistry, Springer-Verlag, Berlin, 2002, 222, 201-258.; Bolton, R. J. Labelled Compd. Radiopharm. 2002, 45 485-528).

These radiopharmaceuticals are distributed and stored in a state in which they are mainly dissolved in physiological saline in the form of an injection, or distilled water for injection. However, when the radioactivity emitted from a radioactive isotope is present as a high dose of radioactivity per unit volume, radiation induces radiolysis in which radiopharmaceuticals are decomposed by radicals produced through the dissociation of water molecules. Due to the characteristics of the radiopharmaceuticals having a short half-life, there is accordingly a need for development of a stabilizing agent capable of preventing radiolysis.

Among the recently developed radiopharmaceuticals, radiopharmaceuticals for diagnosing tumors and brain diseases, which target receptors, also require high specific radioactivity. Therefore, a pH range for purification conditions of radiopharmaceuticals varies, and a pH regulator is thus added during a formulation process. Accordingly, there is a need for development of a stabilizing agent capable of maintaining the stability of radiopharmaceuticals even in a composition to which the pH regulator is added, particularly a stabilizing agent capable of stably maintaining radiopharmaceuticals by inhibiting the radiolysis of the radiopharmaceutical even at a temperature higher than room temperature (15° C. to 25° C.).

### DISCLOSURE

### Technical Problem

Therefore, the present inventors have endeavored to investigate and develop a radiopharmaceutical preparation capable of ensuring stability against radiolysis, and found that, when a vitamin B compound is used as a stabilizing agent, the radiolysis of radiopharmaceuticals is inhibited to stabilize the radiochemical purity of the radiopharmaceuticals at a high level. Therefore, the present invention has been completed based on the facts.

Accordingly, it is an aspect of the present invention to provide a composition for stabilizing a radiopharmaceutical, which includes a vitamin B compound as an active component.

It is another aspect of the present invention to provide a radiopharmaceutical composition including a vitamin B compound as a stabilizing agent for radiopharmaceuticals.

However, the technical objects of the present invention are not limited thereto, and other objects of the present invention which are not stated herein will become more apparent to those of ordinary skill in the art by describing in detail exemplary embodiments thereof.

### Technical Solution

According to an aspect of the present invention, there is provided a composition for stabilizing a radiopharmaceutical, which includes a vitamin B compound as an active component.

According to one embodiment of the present invention, the vitamin B compound may be vitamin B1 or a pharmaceutically acceptable salt thereof, but the present invention is not limited thereto.

According to another embodiment of the present invention, the vitamin B compound may be vitamin B6 or a pharmaceutically acceptable salt thereof, but the present invention is not limited thereto.

According to still another embodiment of the present invention, the radiopharmaceutical may be a compound labeled with ¹⁸F, but the present invention is not limited thereto.

According to yet another embodiment of the present invention, the radiopharmaceutical may be a compound labeled with a metallic radioisotope, but the present invention is not limited thereto.

According to yet another embodiment of the present invention, the metallic radioisotope may be selected from the group consisting of Ga-66, Ga-67, Ga-68, Cu-61, Cu-62, Cu-64, Cu-67, Pb-212, Bi-212, Pd-109, Y-86, Y-90, Co-55, Zr-89, Sr-83, Mn-52, As-72, Sc-44, At-211, Sc-47, In-111, Tc-99m, Lu-177, Ac-225, and Bi-213, but the present invention is not limited thereto.

According to yet another embodiment of the present invention, the composition may inhibit and delay the radiolysis of the radiopharmaceutical, but the present invention is not limited thereto.

According to yet another embodiment of the present invention, the composition may maintain the purity of the radiopharmaceuticals, but the present invention is not limited thereto.

According to yet another embodiment of the present invention, the composition may inhibit or delay the generation of radiochemical impurities, but the present invention is not limited thereto.

According to yet another embodiment of the present invention, the radiolysis may be determined by measuring the radiochemical purity of the radiopharmaceutical, but the present invention is not limited thereto.

According to yet another embodiment of the present invention, the radiopharmaceutical may be a radiopharmaceutical composition including a pH regulator, but the present invention is not limited thereto.

According to yet another embodiment of the present invention, the pH regulator may be a phosphate buffer, but the present invention is not limited thereto.

According to yet another embodiment of the present invention, the phosphate buffer may include a phosphate selected from the group consisting of H₃PO₄, NaH₂PO₄, Na₂HPO₄, Na₃PO₄, KH₂PO₄, K₂HPO₄, K₃HPO₄, (NH₄)H₂PO₄, and (NH₄)₂HPO₄, but the present invention is not limited thereto.

According to another aspect of the present invention, there is provided a radiopharmaceutical composition including a radiopharmaceutical; and a vitamin B compound serving as a stabilizing agent for the radiopharmaceutical.

According to one embodiment of the present invention, the radiopharmaceutical composition may have a radiochemical purity of at least 90% for 2 to 6 hours after the preparation of the radiopharmaceutical composition, as measured at the conditions of a temperature of 40° C. and pH 5 to 8, but the present invention is not limited thereto.

According to another embodiment of the present invention, the radiopharmaceutical composition may have a radiochemical purity of at least 90% for 2 to 4 hours after the preparation of the radiopharmaceutical composition, as measured at the conditions of room temperature and pH 5.5 to 7, but the present invention is not limited thereto.

According to still another embodiment of the present invention, the stabilizing agent may be included at a concentration of 0.1 to 100 mg/mL in the total composition, but the present invention is not limited thereto.

According to yet another embodiment of the present invention, the radiopharmaceutical composition may be a liquid formulation or a lyophilized formulation, but the present invention is not limited thereto.

According to yet another embodiment of the present invention, when the radiopharmaceutical composition is a liquid formulation, the radiopharmaceutical may be a compound labeled with ¹⁸F, but the present invention is not limited thereto.

According to yet another embodiment of the present invention, the radiopharmaceutical composition including the compound labeled with ¹⁸F may further include a pH regulator, but the present invention is not limited thereto.

According to yet another embodiment of the present invention, when the radiopharmaceutical composition is a lyophilized formulation, the radiopharmaceutical may be a compound labeled with a metallic radioisotope, but the present invention is not limited thereto.

According to yet another embodiment of the present invention, the radiopharmaceutical composition including the metallic radioisotope may further include cysteine, but the present invention is not limited thereto.

According to yet another embodiment of the present invention, the radiopharmaceutical composition may be used in positron emission tomography (PET) or single photon emission computed tomography (SPECT).

According to still another aspect of the present invention, there is provided a use of the composition, which includes the vitamin B compound as an active component, for stabilizing the radiopharmaceutical.

According to yet another aspect of the present invention, there is provided a use of the composition, which includes the vitamin B compound as an active component, for producing a stabilizing agent for radiopharmaceuticals.

According to yet another aspect of the present invention, there is provided a method of preparing a stabilized radiopharmaceutical composition, which includes formulating a stabilizing agent including a vitamin B compound as an active component with a radiopharmaceutical.

### Advantageous Effects

The present invention relates to a composition for stabilizing a radiopharmaceutical, which includes a vitamin B compound as an active component; and a radiopharmaceutical composition including the same. The composition includes the vitamin B compound, and thus radiochemical purity can be stabilized by inhibiting the radiolysis of the radioactive compound even at room temperature as well as at a high temperature.

### DESCRIPTION OF DRAWINGS

FIG. 1 shows the results of purifying [¹⁸F] PI-2620 by HPLC.

### BEST MODE

The present invention provides a composition for stabilizing a radiopharmaceutical, which includes a vitamin B compound as an active component.

In the present invention, the vitamin B compound may be a compound selected from the group consisting of B1 (thiamine), B2 (riboflavin), B3 (niacin), B4 (adenine), B5 (pantothenic acid), B6 (pyridoxine), B7 (biotin), B8 (adenosine phosphate), B9 (folic acid), B10 (4-aminobenzoic acid) and B12 (cobalamin), or a salt or derivative thereof, but the present invention is not limited thereto.

According to one specific embodiment, the vitamin B compound may be vitamin B1 or a pharmaceutically acceptable salt thereof, but the present invention is not limited thereto. The vitamin B1 may be represented by the following Formula 1.

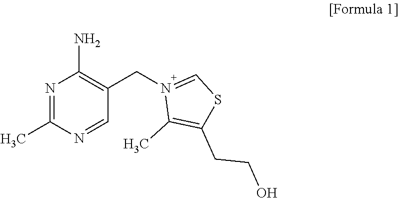

According to another specific embodiment, the vitamin B compound may be vitamin B6 or a pharmaceutically acceptable salt thereof, but the present invention is not limited thereto. The vitamin B6 may be represented by the following Formula 2.

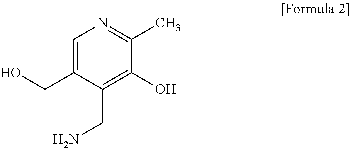

When the stabilization effect of the radiopharmaceutical can be exerted in the range of the vitamin B compound of the present invention as described above, vitamin B derivatives may also be included.

The term “pharmaceutically acceptable salt” used in this specification includes salts derived from pharmaceutically acceptable inorganic acids, organic acids, or bases. Examples of the suitable acids includes hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methane sulfonic acid, formic acid, benzoic acid, malonic acid, gluconic acid, naphthalene-2-sulfonic acid, benzenesulfonic acid, and the like. An acid addition salt may be prepared by a conventional method, for example, by dissolving a compound an excessive amount of an aqueous acid solution and precipitating this salt using a water-miscible organic solvent such as methanol, ethanol, acetone, or acetonitrile. Also, the acid addition salt may be prepared by heating an equimolar amount of the compound and an acid or alcohol in water and drying the mixture by evaporation or filtering the precipitated salt by suction.

Salts derived from the suitable bases include alkali metals such as sodium, potassium, and the like, alkaline earth metals such as magnesium, and the like, and ammonium, and the like, but the present invention is not limited thereto. An alkali metal or alkaline earth metal salt may be prepared, for example, by dissolving a compound in an excessive amount of an alkali metal hydroxide or alkaline earth metal hydroxide solution, filtering an insoluble compound salt, and evaporating and dying the filtrate. In this case, it is particularly suitable to prepare a sodium, potassium, or calcium salt as the metal salt in terms of pharmaceuticals. Also, a silver salt corresponding to the metal salt may be obtained by reacting an alkali metal or alkaline earth metal salt with a suitable silver salt (i.e., silver nitrate).

The term “radiopharmaceutical” used in this specification refers to a radioactive material, such as a radioactive isotope and a compound labeled with the radioactive isotope, which is directly administered to the human body or used in samples such as blood taken from the human body for the purpose of diagnosis and treatment or for the purpose of medical research. Radiopharmaceuticals may be classified for treatment or diagnosis, and in vivo or ex vivo use according to the purpose of use. Diagnostic drugs for in vivo use may be used for the purpose of morphologically diagnosing certain tissues and organs using a radioactive diagnostic drug administered into the body or measuring the functions of the tissues and organs using the nature of the radioactive isotope as a tracer, measuring an amount or a flow expansion time of a certain material (such as blood) in the body, or measuring an uptake rate, a loss rate, a metabolic rate, and the like in each organ.

In the present invention, the radiopharmaceutical may be a compound labeled with ¹⁸F, but the present invention is not limited thereto. For example, the ¹⁸F-labeled compound may be [¹⁸F]PI-2620, [¹⁸F]fluoropropylcarbomethoxytropane, [¹⁸F]fluoromisonidazole, [¹⁸F]fluorothymidine, [¹⁸F]fluoroestradiol, [¹⁸F]fluorodeoxyglucose, [¹⁸F]fluoroDDNP, [¹⁸F]fluorobetaben, [¹⁸F]florbetapir, [¹⁸F]FHBG, [¹⁸F]HX4, [¹⁸F]LBT999, [¹⁸F]flutemetamol, or [¹⁸F]FC119S.

Also, in the present invention, the radiopharmaceutical may be a compound labeled with a metallic radioisotope. The metallic radioisotope may be, for example, selected from the group consisting of Ga-66, Ga-67, Ga-68, Cu-61, Cu-62, Cu-64, Cu-67, Pb-212, Bi-212, Pd-109, Y-86, Y-90, Co-55, Zr-89, Sr-83, Mn-52, As-72, Sc-44, At-211, Sc-47, In-111, Tc-99m, Lu-177, Ac-225, and Bi-213.

The composition of the present invention may be used to stabilize the radiopharmaceutical. Specifically, the composition of the present invention may inhibit or delay the radiolysis of the radiopharmaceutical.

In the present invention, the degree of radiolysis may be determined by measuring a radiochemical purity (RCP) of the radiopharmaceutical. The term “radiochemical purity” refer to a ratio of one target radionuclide to other radionuclides. In this specification, the RCP is expressed as a percentage of activity (% by activity) of a target radioactive compound with respect to the total radioactivity present in a sample.

In the present invention, the radiopharmaceutical stabilized by the vitamin B compound may include a radiopharmaceutical composition including a pH regulator, but the present invention is not limited thereto. According to one specific embodiment, the pH of the radiopharmaceutical composition including the pH regulator may be in a range of pH 5 to 8, pH 6 to 8, pH 7 to 8, or pH 6 to 7.

In the present invention, the pH regulator may be a phosphate buffer, but the present invention is not limited thereto. For example, the phosphate buffer may include a phosphate selected from the group consisting of H₃PO₄, NaH₂PO₄, Na₂HPO₄, Na₃PO₄, KH₂PO₄, K₂HPO₄, K₃HPO₄, (NH₄)H₂PO₄, and (NH₄)₂HPO₄.

According to another aspect of the present invention, the present invention provides a radiopharmaceutical composition including a radiopharmaceutical; and a vitamin B compound serving as a stabilizing agent for the radiopharmaceutical.

In describing the radiopharmaceutical and the vitamin B compound serving as the stabilizing agent as the components of the radiopharmaceutical composition, the contents of the radiopharmaceutical and the vitamin B compound are as described above in the described-above composition for stabilizing the radiopharmaceutical, and thus a description thereof will be omitted.

In the present invention, the radiopharmaceutical composition of the present invention may have a radiochemical purity of at least 90%, at least 91%, at least 92%, at least 93%, at least 94%, at least 95%, at least 96%, at least 97%, or at least 98% for 2 to 6 hours after the preparation of the radiopharmaceutical composition, as measured under the conditions of a temperature of 40° C. and pH 5 to 8, but the present invention is not limited thereto. The radiopharmaceutical composition may include a compound labeled with ¹⁸F. Also, the radiopharmaceutical composition may be a medicinal composition including a pH regulator.

According to one specific embodiment, the radiopharmaceutical composition may have a radiochemical purity of at least 90%, at least 91%, at least 92%, at least 93%, at least 94%, at least 95%, at least 96%, at least 97%, or at least 98% for 2 to 6 hours after the preparation of the radiopharmaceutical composition, as measured under the conditions of a temperature of 40° C. and pH 7 to 8.

According to another specific embodiment, when the radiopharmaceutical composition includes the pH regulator, the radiopharmaceutical composition may have a radiochemical purity of at least 90%, at least 91%, at least 92%, at least 93%, at least 94%, at least 95%, at least 96%, or at least 97% for 2 to 6 hours after the preparation of the radiopharmaceutical composition, as measured under the conditions of a temperature of 40° C. and pH 5 to 6.

Also, in the present invention, the radiopharmaceutical composition of the present invention may have a radiochemical purity of at least 90%, at least 91%, at least 92%, at least 93%, at least 94%, at least 95%, at least 96%, at least 97%, or at least 98% for 2 to 4 hours after the preparation of the radiopharmaceutical composition, as measured under the conditions of room temperature and pH 5.5 to 7, but the present invention is not limited thereto. The radiopharmaceutical composition may include a compound labeled with a metallic radioisotope. Also, the radiopharmaceutical composition may be a medicinal composition including cysteine.

In the present invention, the stabilizing agent may be included at a concentration of 0.1 to 100 mg/mL in the total composition.

In the present invention, the radiopharmaceutical composition of the present invention may be a liquid formulation or a lyophilized formulation, but the present invention is not limited thereto. When the radiopharmaceutical composition is a liquid formulation, the radiopharmaceutical may be a compound labeled with ¹⁸F. In this case, the radiopharmaceutical composition may further include a pH regulator.

Also, when the radiopharmaceutical composition is a lyophilized formulation, the radiopharmaceutical may be a compound labeled with a metallic radioisotope. In this case, the radiopharmaceutical composition may further include cysteine.

### MODE FOR INVENTION

Hereinafter, preferred embodiments of the present invention are presented in order to aid in understanding the present invention. However, it should be understood that the following embodiments are given by way of illustration only to more easily understand the present invention, and are not intended to limit the present invention.

### Synthesis Example: Synthesis of Radiopharmaceuticals (GP-104, GP-105)

### 1-1. Synthesis of tert-butyl (R)-4-(3-(3-(ethoxycarbonyl)piperidin-1-yl)-3-oxopropyl)piperidine-1-carboxylate

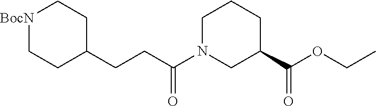 [SMILES: CCOC(=O)[C@@H]1CCCN(C(=O)CCC2CCN(C(=O)OC(C)(C)C)CC2)C1]

3-(1-(tert-butoxycarbonyl)piperidin-4-yl)propanoic acid (1) (367 mg, 2.3 mmol) dissolved in acetonitrile was added to a mixture of ethyl (R)-piperidine-3-carboxylate (2) (500 mg, 1.9 mmol), HBTU (872 mg, 2.3 mmol), HOBt (311 mg, 2.3 mmol), and DIPEA (0.81 mL, 4.6 mmol) at 0° C., and then reacted at room temperature. After 6 hours, 100 mL of each of water and ethyl acetate (EA) was added to separate an organic layer and an aqueous layer. Then, the organic layer was washed with an aqueous 1 M HCl solution (100 mL) and brine (100 mL), filtered over MgSO₄, and then concentrated. The obtained residue was purified by a silica column (Hexane (Hx):EA=1:1) to obtain the final compound (3) (603 mg, 80%).

### 1-2. Synthesis of (R)-1-(3-(1-(tert-butoxycarbonyl)piperidin-4-yl)propanoyl)piperidine-3-carboxylic acid

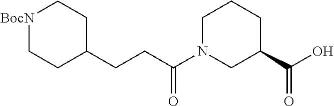 [SMILES: CC(C)(C)OC(=O)N1CCC(CCC(=O)N2CCC[C@@H](C(=O)O)C2)CC1]

The compound (3) (tert-butyl (R)-4-(3-(3-(ethoxycarbonyl)piperidin-1-yl)-3-oxopropyl)piperidine-1-carboxylate, 500 mg, 1.26 mmol) dissolved in tetrahydrofuran (THF, 30 mL) was added to 5 mL of LiOH hydrate (36 mg, 1.51 mmol) at 0° C., and then reacted at room temperature for 3 hours. EA was added to the mixture whose reaction was completed, and optimized to pH 4 by addition of an aqueous 4 M HCl solution. Each of water and EA was added to separate an organic layer and an aqueous layer, and the organic layer was filtered over MgSO₄, and then concentrated. The final compound (4) was obtained as a white solid (350 mg, 75%).

### 1-3. Synthesis of 3-bromo-5-(2-((tert-butyldimethylsilyl)oxy)ethoxy)pyridine

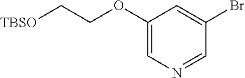 [SMILES: CC(C)(C)[Si](C)(C)OCCOc1cncc(Br)c1]

A mixture of (2-bromoethoxy)(tert-butyl)dimethylsilane (7.4 mL, 34.49 mmol) and K₂CO₃ (7.94 g, 57.5 mmol) was added to a solution in which 3-bromo-5-hydroxypyridine (5) (5.00 g, 28.74 mmol) was dissolved in degassed DMF (30 mL) at room temperature, and then reacted at 80° C. for 15 hours in a sealed reaction tube. After the reaction, the concentrated reaction product was added to each of water (800 mL) and EA (400 mL×3) to separate an organic layer and an aqueous layer, and the organic layer was filtered over MgSO₄, and then concentrated. The residue was purified by a silica column (Hx:EA=5:1) to obtain the final compound (6) as an ivory solid (6.14 g, 64%).

### 1-4. Synthesis of tert-butyl (E)-3-(5-(2-((tert-butyldimethylsilyl)oxy)ethoxy)pyridin-3-yl)acrylate

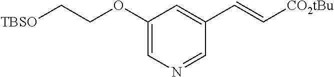 [SMILES: CC(C)(C)OC(=O)/C=C/c1cncc(OCCO[Si](C)(C)C(C)(C)C)c1]

A mixture of tert-butyl acrylate (2.64 mL, 18.05 mmol), K₂CO₃ (3.33 g, 24 mmol), phenylurea (32.8 mg, 0.24 mmol), and palladium diacetate (134 mg, 0.6 mmol) was added to a solution in which 3-bromo-5-(2-((tert-butyldimethylsilyl)oxy)ethoxy)pyridine (6) (4.00 g, 12.04 mmol) was dissolved in degassed DMF (30 mL) at room temperature, and reacted at 130° C. for 12 hours in a sealed reaction tube. After the reaction, the concentrated mixture was added to each of water (400 mL×3) and EA (800 mL) to separate an organic layer and an aqueous layer, and the organic layer was filtered over MgSO₄, and then concentrated. The residue was purified by a silica column (Hx:EA=5:1) to obtain the final compound (7) as an ivory solid (6.14 g, 64%).

### 1-5. Synthesis of tert-butyl (S)-3-(benzyl((R)-1-phenylethyl)amino)-3-(5-(2-((tert-butyldimethylsilyl)oxy)ethoxy)pyridin-3-yl)propanoate

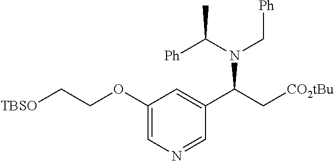 [SMILES: C[C@H](c1ccccc1)N(Cc1ccccc1)[C@@H](CC(=O)OC(C)(C)C)c1cncc(OCCO[Si](C)(C)C(C)(C)C)c1]

A 1.6 M n-BuLi/hexane (15.3 mL, 24.45 mmol) solution was slowly added to a solution in which (R)-(+)-N-benzyl-alpha-methylbenzylamine (4.77 mL, 24.45 mmol) was dissolved in anhydrous THF (50 mL) at −78° C., stirred for 15 minutes, added to a solution in which tert-butyl (E)-3-(5-(2-((tert-butyldimethylsilyl)oxy)ethoxy)pyridin-3-yl)acrylate (7) (5.6 g, 15.28 mmol) was dissolved in anhydrous THF (25 mL), and then reacted at −78° C. for 4 hours. The reaction was terminated by addition of an aqueous NH₄Cl solution, and the concentrated residue was then separated and purified with DCM (1,300 mL) and water (600 mL). Then, the organic layer was filtered over MgSO₄, and then concentrated. The residue was purified by a silica column (Hx:EA=10:1) to obtain the final compound (8) in the form of a brown oil (3.7 g, 54%).

### 1-6. Synthesis of tert-butyl (S)-3-amino-3-(5-(2-((tert-butyldimethylsilyl)oxy)ethoxy)pyridin-3-yl)propanoate

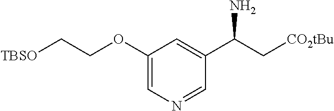 [SMILES: CC(C)(C)OC(=O)C[C@H](N)c1cncc(OCCO[Si](C)(C)C(C)(C)C)c1]

20% palladium hydroxide (752 mg, 20% by weight) was added to a solution in which tert-butyl (S)-3-(benzyl((R)-1-phenylethyl)amino)-3-(5-(2-((tert-butyldimethylsilyl)oxy)ethoxy)pyridin-3-yl)propanoate (8) (3.7 g, 6.36 mmol) was dissolved in MeOH (100 mL) at room temperature under a H₂ (g) atmosphere, and then reacted for 72 hours. The reaction mixture was filtered through Celite, and then concentrated. Thereafter, the concentrated mixture was purified by a silica column (Hx:EA=1:1 to DCM:MeOH=6:1 containing 2% TEA) to obtain the final compound (9) as a brown solid (1.8 g, 71%).

### 1-7. Synthesis of tert-butyl 4-(3-((R)-3-(((S)-3-(tert-butoxy)-1-(5-(2-((tert-butyldimethylsilyl)oxy) ethoxy)pyridin-3-yl)-3-oxopropyl)carbamoyl)piperidin-1-yl)-3-oxopropyl)piperidine-1-carboxylate

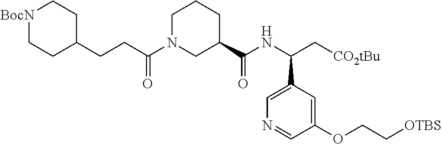 [SMILES: CC(C)(C)OC(=O)C[C@H](NC(=O)[C@@H]1CCCN(C(=O)CCC2CCN(C(=O)OC(C)(C)C)CC2)C1)c1cncc(OCCO[Si](C)(C)C(C)(C)C)c1]

HBTU (1.89 g, 4.99 mmol), TEA (0.7 mL, 4.99 mmol), and (R)-1-(3-(1-(tert-butoxycarbonyl)piperidin-4-yl)propanoyl)piperidine-3-carboxylic acid (4) (1.8 g, 4.54 mmol) were added to a solution in which (R)-1-(3-(1-(tert-butoxycarbonyl)piperidin-4-yl)propanoyl)piperidine-3-carboxylic acid (9) (2.34 g, 4.99 mmol) was dissolved in DMF (40 mL) at room temperature, and then reacted for 12 hours. After the reaction, the mixture was concentrated, and added to each of water (300 mL) and EA (150 mL) to separate an organic layer and an aqueous layer. Then, the organic layer was filtered over MgSO₄, and then concentrated. The residue was purified by a silica column (DCM:MeOH=20:1) to obtain the final compound (10) in the form of a solid (1.4 g, 76%).

### 1-8. Synthesis of tert-Butyl 4-(3-((R)-3-(((S)-3-(tert-butoxy)-1-(5-(2-hydroxyethoxy)pyridin-3-yl)-3-oxopropyl)carbamoyl)piperidin-1-yl)-3-oxopropyl)piperidine-1-carboxylate

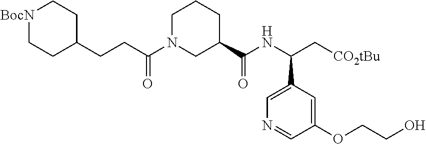 [SMILES: CC(C)(C)OC(=O)C[C@H](NC(=O)[C@@H]1CCCN(C(=O)CCC2CCN(C(=O)OC(C)(C)C)CC2)C1)c1cncc(OCCO)c1]

A 1.0 M TBAF/THF solution (2.8 mL, 2.80 mmol) was added to a solution in which tert-butyl 4-(3-((R)-3-(((S)-3-(tert-butoxy)-1-(5-(2-((tert-butyldimethylsilyl)oxy)ethoxy)pyrid in-3-yl)-3-oxopropyl)carbamoyl)piperidin-1-yl)-3-oxopropyl)piperidine-1-carboxylate (10) (1.4 g, 1.87 mmol) was dissolved in THF (30 mL) at room temperature, and then reacted for 4 hours. After the reaction, the mixture was concentrated, and then purified by a silica column (DCM:MeOH=20:1) to obtain the final compound (11) in the form of a solid (1.1 mg, 94%).

### 1-9. Synthesis of tert-butyl 4-(3-((R)-3-(((S)-3-(tert-butoxy)-3-oxo-1-(5-(2-(tosyloxy)ethoxy)pyridin-3-yl) propyl)carbamoyl)piperidin-1-yl)-3-oxopropyl)piperidine-1-carboxylate

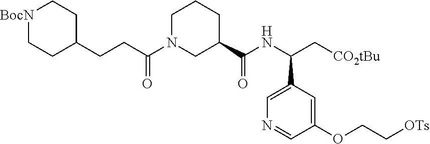 [SMILES: Cc1ccc(S(=O)(=O)OCCOc2cncc([C@H](CC(=O)OC(C)(C)C)NC(=O)[C@@H]3CCCN(C(=O)CCC4CCN(C(=O)OC(C)(C)C)CC4)C3)c2)cc1]

TsCl (7.3 mg, 0.04 mmol) and TEA (6 mg, 0.04 mmol) were added to a solution in which tert-butyl 4-(3-((R)-3-(((S)-3-(tert-butoxy)-1-(5-(2-hydroxyethoxy)pyridin-3-yl)-3-oxopropyl)carbamoyl)piperidin-1-yl)-3-oxopropyl)piperidine-1-carboxylate (11) (20 mg, 0.03 mmol) was dissolved in DCM (1 mL) at room temperature, and then reacted for 12 hours. After the reaction, the mixture was concentrated, and water (150 mL) and DCM (300 mL) were added to the mixture to separate an organic layer. Then, the organic layer was filtered over MgSO₄, and then concentrated. The residue was purified by a silica column (DCM:MeOH=20:1) to obtain the final compound (12) in the form of a solid (20 mg, 83%).

### 1-10. Synthesis of tert-butyl 4-(3-((R)-3-(((S)-1-(5-(2-azidoethoxy)pyridin-3-yl)-3-(tert-butoxy)-3-oxopropyl)carbamoyl)piperidin-1-yl)-3-oxopropyl)piperidine-1-carboxylate

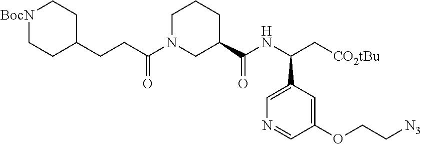 [SMILES: CC(C)(C)OC(=O)C[C@H](NC(=O)[C@@H]1CCCN(C(=O)CCC2CCN(C(=O)OC(C)(C)C)CC2)C1)c1cncc(OCCN=[N+]=[N-])c1]

Sodium azide (12 mg, 0.18 mmol, 3.0 eq) was added to a solution in which tert-butyl 4-(3-((R)-3-(((S)-3-(tert-butoxy)-3-oxo-1-(5-(2-(tosyloxy)ethoxy)pyridin-3-yl)propyl)carbamoyl)piperidin-1-yl)-3-oxopropyl)piperidine-1-carboxylate (12) (50 mg, 0.06 mmol) was dissolved in DMF (0.5 mL), and then reacted at 80° C. for 6 hours. After the reaction, the mixture was cooled, diluted with EA, purified with water and brine, filtered over MgSO₄, and then concentrated to obtain the final compound (13) in the form of a yellow solid (54 mg, 97%).

### 1-11. Synthesis of tert-butyl 4-(3-((R)-3-(((S)-1-(5-(2-aminoethoxy)pyridin-3-yl)-3-(tert-butoxy)-3-oxopropyl)carbamoyl)piperidin-1-yl)-3-oxopropyl)piperidine-1-carboxylate (GP-101)

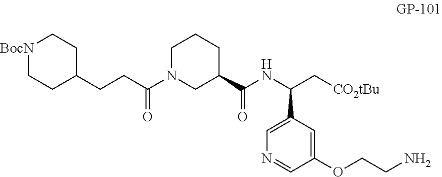 [SMILES: CC(C)(C)OC(=O)C[C@H](NC(=O)[C@@H]1CCCN(C(=O)CCC2CCN(C(=O)OC(C)(C)C)CC2)C1)c1cncc(OCCN)c1]

20% Palladium hydroxide (752 mg, 20% by weight) was added to solution in which (tert-butyl 4-(3-((R)-3-(((S)-1-(5-(2-azidoethoxy)pyridin-3-yl)-3-(tert-butoxy)-3-oxopropyl)carbamoyl)piperidin-1-yl)-3-oxopropyl)piperidine-1-carboxylate (13) (50 mg, 0.08 mmol) was dissolved in MeOH (1 mL) under a H₂ (g) atmosphere at room temperature, and then reacted for 17 hours. The reaction mixture was filtered through Celite, concentrated, and then purified using a silica column (DCM:MeOH=6:1 containing 2% TEA). The final compound obtained in the form of a colorless oil was analyzed by ¹H-NMR (42 mg, 84%).

The ¹H-NMR analysis results of the GP-101 compound are as follows: (using pyridine): 6 8.21-8.18 (m, 2H), 5.38-5.35 (m, 1H), 4.14-3.90 (m, 2H), 3.58-3.44 (m, 2H), 3.10-3.08 (m, 1H), 2.78-2.66 (m, 5H), 2.42-2.28 (m, 3H), 1.91-1.80 (m, 2H), 1.70-1.63 (m, 3H), 1.61-1.55 (m, 2H), 1.44 (s, 11H), 1.35 (s, 11H), 1.16-1.07 (m, 3H), 1.06-0.84 (m, 2H)

### 1-12. Synthesis of tert-butyl 4-(3-((R)-3-(((S)-3-(tert-butoxy)-1-(5-(2-(2-tetra-tert-butyl-DTPA acetamido)ethoxy)pyridin-3-yl)-3-oxopropyl)carbamoyl)piperidin-1-yl)-3-oxopropyl)piperidine-1-carboxylate

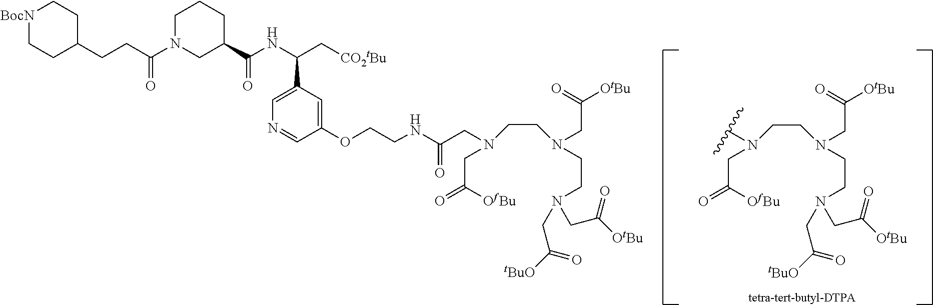

tert-Butyl 4-(3-((R)-3-(((S)-1-(5-(2-aminoethoxy)pyridin-3-yl)-3-(tert-butoxy)-3-oxopropyl)carbamoyl)piperidin-1-yl)-3-oxopropyl)piperidine-1-carboxylate (GP-101) (200 mg, 0.32 mmol), HBTU (144 mg, 0.38 mmol), and HOBt (51 mg, 0.38 mmol) were added to a solution in which 3,6,9-tris(2-(tert-butoxy)-2-oxoethyl)-13,13-dimethyl-11-oxo-12-oxa-3,6,9-triazatetradecanoic acid (235 mg, 0.38 mmol) was dissolved in DMF (1 mL), and DIPEA (132 μL, 0.76 mmol) was added thereto at room temperature under a N2 (g) atmosphere, and reacted at room temperature for 4 hours. The reaction mixture was added to each of water (150 mL) and EA (300 mL) to separate an organic layer and an aqueous layer, and the organic layer was filtered over MgSO₄, and then concentrated. The residue was purified by a silica column (DCM:MeOH=20:1) to obtain the final compound (16) in the form of a solid (236 mg, 60%).

### 1-13. Synthesis of (S)-3-(5-(2-(2-DTPA acetamido)ethoxy)pyridin-3-yl)-3-((R)-1-(3-(piperidin-4-yl)propanoyl)piperidine-3-carboxamido)propanoic acid (GP-104)

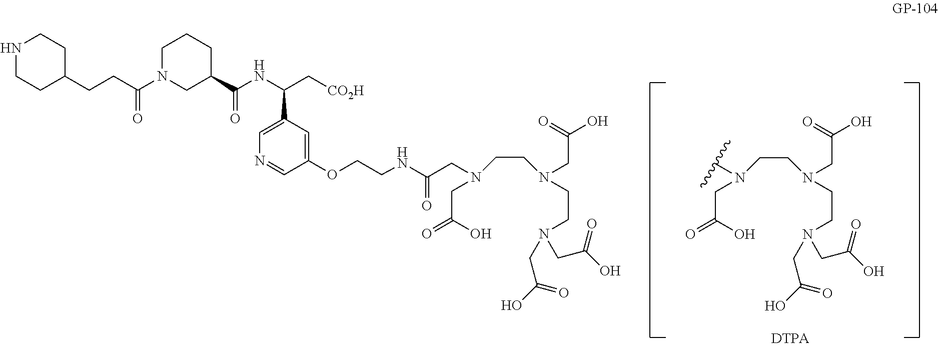

4 mL of 4N-HCl/1,4-dioxane was added to a solution in which tert-butyl 4-(3-((R)-3-(((S)-3-(tert-butoxy)-1-(5-(2-(2-tetra-tert-butyl-DTPA acetamido)ethoxy)pyridin-3-yl)-3-oxopropyl)carbamoyl)piperidin-1-yl)-3-oxopropyl)piperidine-1-carboxylate (16) (210 mg, 0.17 mmol) was dissolved in DCM (1 mL) at room temperature, and then reacted for 48 hours under the same atmosphere. The reaction mixture was concentrated and purified by DCM slurry to obtain the solid final compound (60 mg, 41%). Mass spectrometry (Agilent 6120. Single quadrupole) [M+H] 851.4

The ¹H-NMR analysis results of GP-104 are as follows: (BRUKER, MeOD, 400 MHz NMR): 6 8.59-8.53 (m, 2H), 8.39-8.35 (m, 1H), 5.46-5.41 (m, 1H), 4.40-4.34 (m, 7H), 4.31-4.23 (m, 6H), 3.85-3.79 (m, 2H), 3.75-3.68 (m, 6H), 3.68-3.58 (m, 4H), 3.42-3.35 (m, 3H), 3.29-3.19 (m, 4H), 3.07-2.96 (m, 3H), 2.89-2.88 (m, 1H), 2.61-2.46 (m, 2H), 2.04-1.97 (m, 3H), 1.84-1.50 (m, 5H), 1.50-1.30 (m, 2H)

### 1-14. Synthesis of di-tert-butyl 2,2′-((2-((2-((5-((S)-3-(tert-butoxy)-1-((R)-1-(3-(1-(tert-butoxycarbonyl)piperidin-4-yl)propanoyl)piperidine-3-carboxamido)-3-oxopropyl)pyridin-3-yl)oxy)ethyl)amino)-2-oxoethyl)azanediyl)diacetate

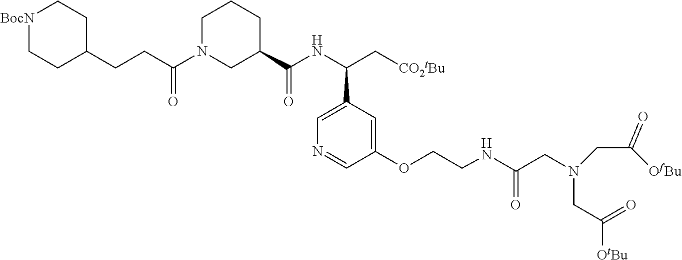

tert-butyl 4-(3-((R)-3-(((S)-1-(5-(2-aminoethoxy)pyridin-3-yl)-3-(tert-butoxy)-3-oxopropyl)carbamoyl)piperidin-1-yl)-3-oxopropyl)piperidine-1-carboxylate (GP-101) (200 mg, 0.32 mmol), HBTU (144 mg, 0.38 mmol), and HOBt (51 mg, 0.38 mmol) were added to a solution in which bis(2-(tert-butoxy)-2-oxoethyl)glycine (115 mg, 0.38 mmol) was dissolved in DMF (1 mL), and DIPEA (132 μL, 0.76 mmol) was added thereto at room temperature under a N2 (g) atmosphere, and then reacted at room temperature for 4 hours. The reaction mixture was added to each of water (150 mL) and EA (300 mL) to separate an organic layer and an aqueous layer, and the organic layer was filtered over MgSO₄, and then concentrated. The obtained residue was purified by a silica column (DCM:MeOH=20:1) to obtain the final compound (17) in the form of a solid (190 mg, 65%).

### 1-15. Synthesis of 2,2′-((2-((2-((5-((S)-2-carboxy-1-((R)-1-(3-(piperidin-4-yl)propanoyl)piperidine-3-carboxamido)ethyl)pyridin-3-yl)oxy)ethyl)amino)-2-oxoethyl)azanediyl)diacetic acid (GP-105)

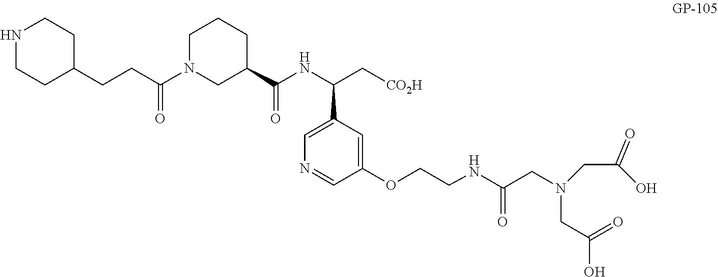 [SMILES: O=C(O)C[C@H](NC(=O)[C@@H]1CCCN(C(=O)CCC2CCNCC2)C1)c1cncc(OCCNC(=O)CN(CC(=O)O)CC(=O)O)c1]

4 mL of 4 N-HCl/1,4-dioxane was added to a solution in which di-tert-butyl 2,2′-((2-((2-((5-((S)-3-(tert-butoxy)-1-((R)-1-(3-(1-(tert-butoxycarbonyl)piperidin-4-yl)propanoyl)piperidine-3-carboxamido)-3-oxopropyl)pyridin-3-yl)oxy)ethyl)amino)-2-oxoethyl)azanediyl)diacetate (17) (180 mg, 0.20 mmol) was dissolved in DCM (1 mL) at room temperature, and then reacted for 48 hours under the same atmosphere. The reaction mixture was concentrated and purified by DCM slurry to obtain the solid final compound (65 mg, 51%). The reaction mixture was concentrated and purified by DCM slurry to obtain the solid final compound (20 mg, 39%).

The solid GP-105 (50 mg) was dissolved in ACN/water (5 mL/5 mL), and 0.5 mL of the resulting mixture was transferred to each of 20 vials, and lyophilized for 3 hours under the condition of −40° C. After the vacuum was activated, the mixture was lyophilized for a day under the condition of −40° C., for a day under the condition of 0° C., and for 4 hours under the condition of 25° C. Each of the vials was covered with a rubber stopper, and the vacuum was released. The vials containing 2.5 mg of the dried GP-105 per one vial were covered with an aluminum stopper, and the stored under the condition of −20° C. Mass spectrometry (Agilent 6120. Single quadrupole) [M+H] 649.3

The ¹H-NMR analysis results of GP-105 are as follows: (BRUKER, MeOD, 400 MHz NMR): δ 8.57-8.52 (m, 2H), 8.33-8.28 (m, 1H), 5.45-5.38 (m, 1H), 4.39-4.25 (m, 9H), 3.77-3.68 (m, 3H), 3.42-3.37 (m, 3H), 3.01-2.98 (m, 5H), 2.88-2.84 (m, 1H), 2.54-2.46 (m, 3H), 2.05 (s, 1H), 2.04-1.98 (m, 3H), 1.72-1.39 (m, 9H)

### Example 1: Decrease in Purity of Conventional Stabilizing Agent-Containing Radiopharmaceutical According to Increase in Temperature

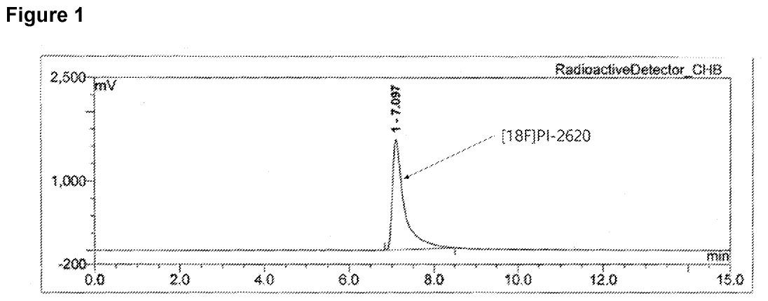

Fluoride ([¹⁸F]) and 2 mg of a precursor were dissolved in dimethyl sulfoxide, and the reaction mixture was fluorinated using an SN2 reaction at a temperature of 100° C. or higher. Then, a protecting group was hydrolyzed with sulfuric acid, and the mixture was neutralized with sodium hydroxide, purified using high-performance liquid chromatography (HPLC) (purification conditions: Luna C18 250×10 mm, phosphate buffer:acetonitrile=70:30 (v/v), 5.0 mL/min, UV=254 nm), and then formulated (formulation composition: 100 mg of sodium ascorbate, 2 nM phosphate buffer, and 10% ethanol) to obtain the [¹⁸F]PI-2620 compound having a radiochemical purity of 100% (FIG. 1).

[¹⁸F]PI-2620 was formulated with ethanol, ascorbic acid, and a phosphate buffer, and the radiochemical purity of the radiopharmaceutical composition was then measured at the elapsed times of 0 hour, 2 hours, 4 hours, and 6 hours at a temperature of 40° C. using HPLC (analysis conditions: Luna C18 250×4.6 mm, phosphate buffer:acetonitrile=60:40 (v/v), 1.0 mL/min, UV=254 nm).

TABLE 1

| | |
|---|---|
| Stabilizing agent | Na ascorbate |

| | | |
|---|---|---|
| 0 hr | Purity | 99.05 |
| | PH (paper) | 6 |
| 2 hr | Purity | 95.53 |
| | PH (paper) | 6 |
| 4 hr | Purity | 93.77 |
| | PH (paper) | 6 |
| 6 hr | Purity | 90.04 |
| | PH (paper) | 6 |

As shown in Table 1, it can be seen that sodium ascorbate known in the art as the stabilizing agent for radiopharmaceuticals has a very low radiochemical purity due to radiolysis under the condition of a high temperature of 40° C. according to the pharmaceutical evaluation criteria of the Pharmaceutical Affairs Act (Notification No. 2019-132 published for the guidelines on stability testing of pharmaceutical products by the Ministry of Food and Drug Safety).

### Example 2: Confirmation of Change in Effect According to Increase in Use Amount of Conventional Stabilizing Agent

[¹⁸F]PI-2620 was obtained as a compound having a radiochemical purity of 100% in the same manner as in Example 1. To further confirm the stability of the compound, 2 equivalents of sodium ascorbate used in Example 1 was added during a formulation process (formulation composition: 200 mg of sodium ascorbate, a 2 nM phosphate buffer, and 10% ethanol), and the radiochemical purity of the radiopharmaceutical composition was then measured in the same manner as in Example 1 at the elapsed times of 0 hour, 2 hours, 4 hours, and 6 hours at a temperature of 40° C. using high-performance liquid chromatography (HPLC)

TABLE 2

| | |
|---|---|
| Stabilizing agent | 2x Na ascorbate |

| | | |
|---|---|---|
| 0 hr | Purity | 99.47 |
| | PH (paper) | 6 |
| 2 hr | Purity | 96.95 |
| | PH (paper) | 6 |
| 4 hr | Purity | 95.25 |
| | PH (paper) | 6 |
| 6 hr | Purity | 93.00 |
| | PH (paper) | 6 |

As shown in Table 2, it can be seen the radiochemical purity after the elapsed time of 6 hours was slightly increased to 93.00% even when the amount of sodium ascorbate as the conventional stabilizing agent was doubled.

### Example 3: Confirmation of Cause of Decrease in Radiochemical Purity

[¹⁸F]PI-2620 was obtained as a compound having a radiochemical purity of 100% in the same manner as in Example 1. To check the causes of a decrease in radiochemical purity, the radiochemical purity of the radiopharmaceutical composition was then measured in the same manner as in Example 1 at the elapsed times of 0 hour, 2 hours, 4 hours, and 6 hours at a temperature of 40° C. under three formulation conditions (Condition 1: ethanol and water for injection (formulation composition: 10% ethanol); Condition 2: ethanol and a phosphate buffer (formulation composition: a 2 nM phosphate buffer and 10% ethanol); Condition 3: ethanol and a 2×phosphate buffer (formulation composition: a 4 nM phosphate buffer and 10% ethanol)) using high-performance liquid chromatography (HPLC).

TABLE 3

| | | | Ethanol + | Ethanol + 2x |
|---|---|---|---|---|
| | | | phosphate | phosphate |
| | | | buffer | buffer |
| | | | (H₃PO₄ + | (H₃PO₄ + |
| | | Ethanol | NaH₂PO₄) | NaH₂PO₄) |
| Stabilizing agent | | (Condition 1) | (Condition 2) | (Condition 3) |
| 0 hr | Purity | 98.93 | 100 | 99.34 |
| | PH (paper) | 6 | 6 | 6 |
| 2 hr | Purity | 99.64 | 96.30 | 94.24 |
| | PH (paper) | 6 | 6 | 6 |
| 4 hr | Purity | 99.09 | 94.80 | 90.54 |
| | PH (paper) | 6 | 6 | 6 |
| 6 hr | Purity | 98.75 | 90.80 | 85.33 |
| | PH (paper) | 6 | 6 | 6 |

As shown in Table 3, it can be seen that [¹⁸F]PI-2620 was not decomposed at a high temperature due to the characteristics of the compound because the initial radiochemical purity was well maintained for 6 hours when [¹⁸F]PI-2620 was formulated with ethanol and water for injection.

Also, it can be seen that the radiochemical purity was decreased to 90.80% after the elapsed time of 6 hours when the pH regulator (H₃PO₄, NaH₂PO) was added, and radiolysis was accelerated as the radiochemical purity was decreased to 85.33% even when the content of the pH regulator was doubled.

### Example 4: Evaluation of Stabilization Effect of Novel Stabilizing Agent

[¹⁸F]PI-2620 was obtained as a compound having a radiochemical purity of 98% or more in the same manner as in Example 1. As a novel stabilizing agent, vitamin B1 (VB1), vitamin B6 (VB6), vitamin B8 (VB8), vitamin B10 (VB10), or vitamin B12 (VB12) (formulation composition: 10% ethanol and a 50 mg/mL vitamin concentration) was added to ethanol and water for injection, and the radiochemical purity of the radiopharmaceutical composition was measured in the same manner as in Example 1 at the elapsed times of 0 hour, 2 hours, 4 hours, and 6 hours at a temperature of 40° C. using high-performance liquid chromatography (HPLC). A preparation including no vitamin B was used as the control.

TABLE 4

| Stabilizing agent | | Control | VB1 | VB6 | VB8 | VB10 | VB12 |
|---|---|---|---|---|---|---|---|
| 0 hr | Purity | 98.93 | 98.92 | 99.16 | 98.92 | 99.15 | 98.33 |
| | PH (paper) | 6 | 8 | 8 | 6 | 7 | 6 |
| 2 hr | Purity | 99.64 | 98.97 | 99.2 | 99.14 | 98.71 | 98.99 |
| | PH (paper) | 6 | 7 | 7 | 6 | 6 | 7.5 |
| 4 hr | Purity | 99.09 | 99.1 | 99.15 | 98.8 | 98.83 | 98.7 |
| | PH (paper) | 6 | 7 | 7 | 6 | 7 | 6.5 |
| 6 hr | Purity | 98.94 | 98.94 | 99.04 | 98.2 | 98.73 | 98.29 |
| | PH (paper) | 6 | 7 | 7 | 6 | 7 | 6.5 |

As shown in Table 4, it can be seen that, even when five vitamins were added, the initial radiochemical purity was well maintained for 6 hours, which was identical when [¹⁸F]PI-2620 was formulated with ethanol and water for injection.

### Example 5: Evaluation of Stabilization Effect of Novel Stabilizing Agent

[¹⁸F]PI-2620 was obtained as a compound having a radiochemical purity of 98% or more in the same manner as in Example 1, and formulated with ethanol and a phosphate buffer (formulation composition: a 2 nM phosphate buffer and 10% ethanol). As a novel stabilizing agent, vitamin B1 (VB1), vitamin B6 (VB6), vitamin B8 (VB8), or vitamin B10 (VB10) was added at a concentration of 50 mg/mL, and the radiochemical purity of the radiopharmaceutical composition was measured in the same manner as in Example 1 at the elapsed times of 0 hour, 2 hours, 4 hours, and 6 hours at a temperature of 40° C. using high-performance liquid chromatography (HPLC). A preparation in which sodium ascorbate was added to the same concentrations of ethanol and phosphate buffer was used as the control.

TABLE 5

| Stabilizing agent | | Control | VB1 | VB6 | VB8 | VB10 |
|---|---|---|---|---|---|---|
| 0 hr | Purity | 99.05 | 98.79 | 98.74 | 98.06 | 98.01 |
| | PH (paper) | 6 | 5 | 5 | 6 | 6 |
| 2 hr | Purity | 95.53 | 98.46 | 98.25 | 95.43 | 94.44 |
| | PH (paper) | 6 | 5.5 | 5 | 6 | 6 |
| 4 hr | Purity | 93.77 | 98.97 | 97.78 | 92.46 | 91.25 |
| | PH (paper) | 6 | 5.5 | 5 | 6 | 6 |
| 6 hr | Purity | 90.04 | 98.94 | 97.33 | 87.76 | 86.09 |
| | PH (paper) | 6 | 5.5 | 5 | 6 | 6 |

As shown in Table 5, it can be seen that the purity was decreased to 90.04% when [¹⁸F]PI-2620 included sodium ascorbate and a phosphate buffer, and the purity rather slightly decreased to 90% or less (87.76% and 86.09%, respectively) at the elapsed time of 6 hours when vitamin B8 (VB8) and vitamin B10 (VB10) were added.

When vitamin B1 (VB1) and vitamin B6 (VB6) were used as the stabilizing agent, the radiopharmaceutical composition had a radiochemical purity of 98.94% and 97.33%, respectively, even after the elapsed time of 6 hours under the condition of a high temperature of 40° C. As a result, it can be seen that the vitamin B group compound was able to be prevent radiolysis at a high temperature.

### Example 6: Confirmation of Radiochemical Purity According to Content of Vitamin B Compound

A radiopharmaceutical composition was obtained in the same manner as in Example 5, except that the compounds of vitamin B1 (VB1) and vitamin B6 (VB6) were dissolved to a concentration of 0.78 mg/mL, and the radiochemical purity of the radiopharmaceutical composition was measured. The results are shown in Table 6 below.

TABLE 6

| | | |
|---|---|---|
| Stabilizing agent | VB1 | VB6 |

| | | | |
|---|---|---|---|
| 0 hr | Purity | 99.66 | 99.31 |
| | PH (paper) | 6 | 6 |
| 2 hr | Purity | 99.21 | 98.32 |
| | PH (paper) | 6 | 6 |
| 4 hr | Purity | 97.63 | 94.4 |
| | PH (paper) | 6 | 6 |
| 6 hr | Purity | 97.12 | 92.17 |
| | PH (paper) | 6 | 6 |

As shown in Table 6, it can be seen that the radiopharmaceutical composition had a high radiochemical purity even when a low concentration of the stabilizing agent was included in the radiopharmaceutical composition, and, particularly, had a high radiochemical purity of 97.12% at a low concentration of 0.78 mg/mL when the radiopharmaceutical composition included vitamin B1.

### Example 7: Preparation of ^{99m}Tc-Labeled Radiopharmaceutical (GP-104)-Containing Preparation (Including Cysteine)

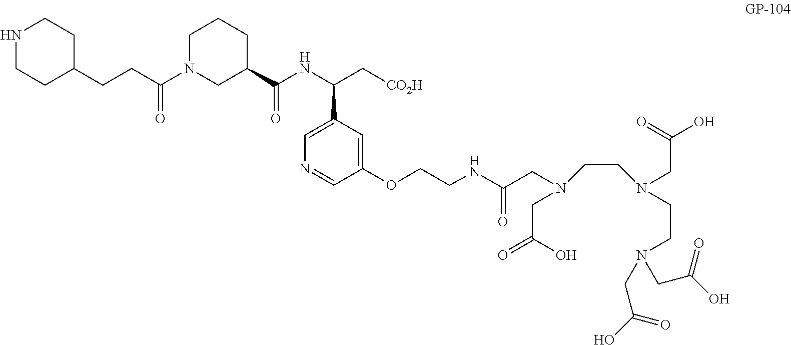 [SMILES: O=C(O)C[C@H](NC(=O)[C@@H]1CCCN(C(=O)CCC2CCNCC2)C1)c1cncc(OCCNC(=O)CN(CCN(CCN(CC(=O)O)CC(=O)O)CC(=O)O)CC(=O)O)c1]

2 mL of Tc-99m was added dropwise to a lyophilization vial containing 0.1 mg of GP-104, 15 μg of SnCl₂, and 20 μg (165 nmol) of cysteine, and left at room temperature for 10 minutes, and the reaction was terminated by adjusting the pH of the mixture to pH 7 using a NaOH solution. The labeling purity was determined using HPLC (analysis conditions: Luna C18 250×4.6 mm, phosphate buffer:acetonitrile (100:0, 0-5 minutes; 80:20, 5-10 minutes; 80:20, 17 minutes; 100:0, 17.1 minutes; 100:0. 20 minutes), 1.0 mL/min, UV=280 nm).

### Example 8: Evaluation of Stability of ^{99m}Tc-Labeled Radiopharmaceutical (GP-104)-Containing Preparation Under Acidic and Basic Conditions

HCl and NaOH were further added dropwise to the solution of Example 7 to adjust the final pH of the solution, followed by stability evaluation under the acidic (pH 5.5) and basic (pH 8.5) conditions. Then, the labeling purity was determined in the same manner as in Example 7 using HPLC.

TABLE 7

| | pH 5.5 | pH 7 | pH 8.5 |
|---|---|---|---|
| 0 hr | 95.26 | 95.26 | 95.26 |
| 2 hr | 91.45 | 91.19 | 79.83 |
| 4 hr | 89.28 | 91.61 | 51.77 |

As shown in Table 7, it was confirmed that the radiochemical purity was maintained at 90% or more for 4 hours at pH 7 in a solution including 165 nmol cysteine, but the radiopharmaceutical composition had a radiochemical purity of 90% or less at the elapsed time of 4 hours under the acidic conditions and had a radiochemical purity of 80% or less from the elapsed time of 2 hours under the basic conditions.

### Example 9: Preparation of ^{99m}Tc-Labeled Radiopharmaceutical (GP-104)-Containing Preparation (Including Cysteine and VB1)

2 mL of Tc-99m was added dropwise to a lyophilization vial containing 0.1 mg of radiopharmaceutical GP-104, 15 μg of SnCl₂, 10 μg (82.5 nmol) of cysteine, and 22 μg (82.5 nmol) of VB1, and left at room temperature for 10 minutes, and the reaction was terminated by adjusting the pH of the mixture to pH 7 using a NaOH solution. Then, the labeling purity was determined in the same manner as in Example 7 using HPLC.

### Example 10: Evaluation of Stability of ^{99m}Tc-Labeled Radiopharmaceutical (GP-104)-Containing Preparation Under Acidic and Basic Conditions

HCl and NaOH were further added dropwise to the solution of Example 9 to adjust the final pH of the solution, followed by stability evaluation under the acidic (pH 5.5) and basic (pH 8.5) conditions. Then, the labeling purity was determined in the same manner as in Example 7 using HPLC.

TABLE 8

| | pH 5.5 | pH 7.0 | pH 8.5 |
|---|---|---|---|
| 0 hr | 100 | 100 | 100 |
| 2 hr | 92.72 | 98.63 | 83.08 |
| 4 hr | 91.20 | 98.18 | 80.97 |

As shown in Table 8, it was confirmed that the radiochemical purity was maintained at a high purity level of 98% or more for 4 hours at pH 7 in a solution including cysteine corresponding to half the moles of cysteine of Example 7 and VB6 of the same moles, and the radiopharmaceutical composition had a radiochemical purity of 90% or more at the elapsed time of 4 hours under the acidic conditions and had a radiochemical purity of approximately 80% from the elapsed time 2 hours to 4 hours under the basic conditions.

### Example 11: Preparation of ^{99m}Tc-Labeled Radiopharmaceutical (GP-104)-Containing Preparation (Including Cysteine and VB6)

2 mL of Tc-99m was added dropwise to a lyophilization vial containing 0.1 mg of GP-104, 15 μg of SnCl₂, 10 μg (82.5 nmol) of cysteine, and 17 μg (82.5 nmol) of VB6, and left at room temperature for 10 minutes, and the reaction was terminated by adjusting the pH of the mixture to pH 7 using a NaOH solution. Then, the labeling purity was determined in the same manner as in Example 7 using HPLC.

### Example 12: Evaluation of Stability of ^{99m}Tc-Labeled Radiopharmaceutical (GP-104)-Containing Preparation Under Acidic and Basic Conditions

HCl and NaOH were further added dropwise to the solution of Example 11 to adjust the final pH of the solution, followed by stability evaluation under the acidic (pH 5.5) and basic (pH 8.5) conditions. Then, the labeling purity was determined in the same manner as in Example 7 using HPLC.

TABLE 9

| | pH 5.5 | pH 7.0 | pH 8.5 |
|---|---|---|---|
| 0 hr | 97.24 | 97.24 | 97.24 |
| 2 hr | 90.29 | 95.61 | 95.61 |
| 4 hr | 90.65 | 96.25 | 91.19 |

As shown in Table 9, it was confirmed that the radiochemical purity was maintained at a high purity level of 96% or more for 4 hours at pH 7 in a solution including cysteine corresponding to half the moles of cysteine of Example 7 and VB6 of the same moles, and the radiopharmaceutical composition had a radiochemical purity of 90% or more at the elapsed time of 4 hours even under the acidic and basic conditions.

### Example 13: Preparation of ^{99m}Tc-Labeled Radiopharmaceutical (GP-105)-Containing Preparation (Including Cysteine)

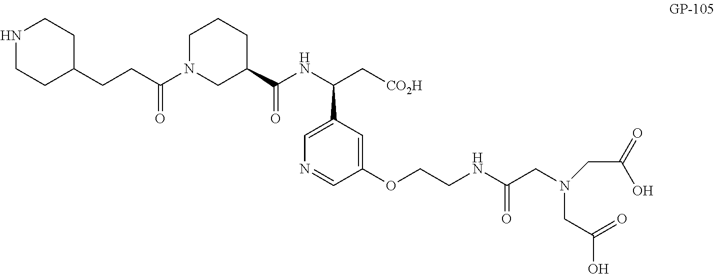 [SMILES: O=C(O)C[C@H](NC(=O)[C@@H]1CCCN(C(=O)CCC2CCNCC2)C1)c1cncc(OCCNC(=O)CN(CC(=O)O)CC(=O)O)c1]

2 mL of Tc-99m was added dropwise to a lyophilization vial containing 0.1 mg of GP-105, 15 μg of SnCl₂, and 20 μg (165 nmol) of cysteine, and left at room temperature for 10 minutes, and the reaction was terminated by adjusting the pH of the mixture to pH 7 using a NaOH solution. Then, the labeling purity was determined in the same manner as in Example 7 using HPLC.

### Example 14: Evaluation of Stability of ^{99m}Tc-Labeled Radiopharmaceutical (GP-105)-Containing Preparation Under Acidic and Basic Conditions

HCl and NaOH were further added dropwise to the solution of Example 13 to adjust the final pH of the solution, followed by stability evaluation under the acidic (pH 5.5) and basic (pH 8.5) conditions. Then, the labeling purity was determined in the same manner as in Example 7 using HPLC.

TABLE 10

| | pH 5.5 | pH 7 | pH 8.5 |
|---|---|---|---|
| 0 hr | 100 | 100 | 100 |
| 2 hr | 85.49 | 94.88 | 50.64 |
| 4 hr | 82.11 | 85.55 | 23.98 |

As shown in Table 10, it was confirmed that the radiochemical purity was maintained at 90% or more for 2 hours at pH 7 in a solution including 165 nmol cysteine, and the radiopharmaceutical composition had a radiochemical purity of 90% or less from the elapsed time of 2 hours under the acidic conditions and had a radiochemical purity of 50% from the elapsed time of 2 hours and a very low radiochemical purity of approximately 23% at the elapsed time of 4 hours under the basic conditions.

### Example 15: Preparation of ^{99m}Tc-Labeled Radiopharmaceutical (GP-105)-Containing Preparation (Including Cysteine and VB1)

2 mL of Tc-99m was added dropwise to a lyophilization vial containing 0.1 mg of GP-105, 15 μg of SnCl₂, 10 μg (82.5 nmol) of cysteine, and 22 μg (82.5 nmol) of VB1, and left at room temperature for 10 minutes, and the reaction was terminated by adjusting the pH of the mixture to pH 7 using a NaOH solution. Then, the labeling purity was determined in the same manner as in Example 7 using HPLC.

### Example 16: Evaluation of Stability of ^{99m}Tc-Labeled Radiopharmaceutical (GP-105)-Containing Preparation Under Acidic and Basic Conditions

HCl and NaOH were further added dropwise to the solution of Example 15 to adjust the final pH of the solution, followed by stability evaluation under the acidic (pH 5.5) and basic (pH 8.5) conditions. Then, the labeling purity was determined in the same manner as in Example 7 using HPLC.

TABLE 11

| | pH 5.5 | pH 7 | pH 8.5 |
|---|---|---|---|
| 0 hr | 100 | 100 | 100 |
| 2 hr | 100 | 100 | 94.13 |
| 4 hr | 98.63 | 94.88 | 90.17 |

As shown in Table 11, it was confirmed that the radiochemical purity was maintained at a purity level of 94% or more for 4 hours at pH 7 in a solution including cysteine corresponding to half the moles of cysteine of Example 13 and VB1 of the same moles, and the radiopharmaceutical composition had a high radiochemical purity of 98% or more at the elapsed time of 4 hours under the acidic conditions and had a radiochemical purity of 90% or more at the elapsed time of 4 hours under the basic conditions.

### Example 17: Preparation of ^{99m}Tc-Labeled Radiopharmaceutical (GP-105)-Containing Preparation (Including Cysteine and VB6)

2 mL of Tc-99m was added dropwise to a lyophilization vial containing 0.1 mg of GP-105, 15 μg of SnCl₂, 10 μg (82.5 nmol) of cysteine, and 17 μg (82.5 nmol) of VB6, and left at room temperature for 10 minutes, and the reaction was terminated by adjusting the pH of the mixture to pH 7 using a NaOH solution. Then, the labeling purity was determined in the same manner as in Example 7 using HPLC.

### Example 18: Evaluation of Stability of ^{99m}Tc-Labeled Radiopharmaceutical (GP-105)-Containing Preparation Under Acidic and Basic Conditions

HCl and NaOH were further added dropwise to the solution of Example 17 to adjust the final pH of the solution, followed by stability evaluation under the acidic (pH 5.5) and basic (pH 8.5) conditions. Then, the labeling purity was determined in the same manner as in Example 7 using HPLC.

TABLE 12

| | pH 5.5 | pH 7 | pH 8.5 |
|---|---|---|---|
| 0 hr | 100 | 100 | 100 |
| 2 hr | 97.24 | 98.56 | 92.21 |
| 4 hr | 95.61 | 98.2 | 89.62 |

As shown in Table 12, it was confirmed that the radiochemical purity was maintained at a high purity level of 98% or more for 4 hours at pH 7 in a solution including cysteine corresponding to half the moles of cysteine of Example 13 and VB6 of the same, and maintained at a purity level of 95% or more at the elapsed time of 4 hours even under the acidic conditions, but the radiopharmaceutical composition had a radiochemical purity of 90% or less at the elapsed time of 4 hours under the basic conditions.

The above description of the present invention has been given by way of illustration only. Therefore, those skilled in the art to which the present invention pertains will appreciate that the present invention can be embodied in other specific forms without changing the technical spirit or essential features of the present invention.

Therefore, it should be understood that the embodiments described above are for the purpose of illustration only, and not intended to be limiting in all respects.

### INDUSTRIAL APPLICABILITY

The present invention relates to a composition for stabilizing a radiopharmaceutical, which includes a vitamin B compound as an active component; and a radiopharmaceutical composition including the same. The composition includes the vitamin B compound, and thus radiochemical purity can be stabilized by inhibiting the radiolysis of the radioactive compound even at room temperature as well as at a high temperature. Therefore, the present invention is industrially applicable.

1-22. (canceled)

23. A method of preparing a stabilized radiopharmaceutical composition, comprising:
formulating a stabilizing agent comprising a vitamin B compound as an active component with a radiopharmaceutical.

24. The method of claim 23, wherein the stabilized radiopharmaceutical composition has a radiochemical purity of at least 90% for 2 to 6 hours after preparation of the radiopharmaceutical composition, as measured at conditions of a temperature of 40° C. and pH 5 to 8.

25. The method of claim 23, wherein the stabilized radiopharmaceutical composition has a radiochemical purity of at least 90% for 2 to 4 hours after preparation of the radiopharmaceutical composition, as measured at conditions of room temperature and pH 5.5 to 7.

26. The method of claim 23, wherein the stabilizing agent is included at a concentration of 0.1 to 100 mg/mL in the total stabilized radiopharmaceutical composition.

27. The method of claim 23, wherein the stabilized radiopharmaceutical composition is a liquid formulation or a lyophilized formulation.

28. The method of claim 27, wherein the radiopharmaceutical is a compound labeled with ¹⁸F when the stabilized radiopharmaceutical composition is a liquid formulation.

29. The method of claim 28, wherein the stabilized radiopharmaceutical composition further comprises a pH regulator.

30. The method of claim 27, wherein the radiopharmaceutical is a compound labeled with a metallic radioisotope when the stabilized radiopharmaceutical composition is a lyophilized formulation.

31. The method of claim 30, wherein the stabilized radiopharmaceutical composition further comprises cysteine.

32. The method of claim 23, wherein the vitamin B compound is vitamin B1 or a pharmaceutically acceptable salt thereof.

33. The method of claim 23, wherein the vitamin B compound is vitamin B6 or a pharmaceutically acceptable salt thereof.

34. The method of claim 30, wherein the metallic radioisotope is selected from the group consisting of Ga-66, Ga-67, Ga-68, Cu-61, Cu-62, Cu-64, Cu-67, Pb-212, Bi-212, Pd-109, Y-86, Y-90, Co-55, Zr-89, Sr-83, Mn-52, As-72, Sc-44, At-211, Sc-47, In-111, Tc-99m, Lu-177, Ac-225, and Bi-213.

35. The method of claim 23, wherein the stabilizing agent inhibits or delays the radiolysis of the radiopharmaceutical.

36. The method of claim 35, wherein the radiolysis is determined by measuring a radiochemical purity of the radiopharmaceutical.

37. The method of claim 29, wherein the pH regulator is a phosphate buffer.

38. The method of claim 37, wherein the phosphate buffer comprises a phosphate selected from the group consisting of H₃PO₄, NaH₂PO₄, Na₂HPO₄, Na₃PO₄, KH₂PO₄, K₂HPO₄, K₃HPO₄, (NH₄)H₂PO₄, and (NH₄)₂HPO₄.
